(19) United States
(12) Patent Application Publication
(10) Pub. No.: US 2026/0055038 A1
(43) Pub. Date: Feb. 26, 2026

(54) SLURRY CONTAINING AN AROMATIC HYDROCARBON AND METHODS THEREOF

(71) Applicant: Solid Power Operating, Inc., Louisville, CO (US)

(72) Inventors: Morgan MORRONI, Denver, CO (US); Ian A. MORRISSEY, Boulder, CO (US); Clint R. BICKMORE, Louisville, CO (US)

(21) Appl. No.: 19/309,055

(22) Filed: Aug. 25, 2025

Related U.S. Application Data
(60) Provisional application No. 63/686,643, filed on Aug. 23, 2024.

(51) Int. Cl. C07C 15/38; H01M 4/04; H01M 4/139; H01M 10/0562

(52) U.S. Cl. CPC C07C 15/38; H01M 4/0404; H01M 4/139; H01M 10/0562; H01M 2300/0068

(57) ABSTRACT

Methods for making solid-state electrochemical battery cell layers include forming a slurry that includes a solid-state electrolyte material and an aromatic hydrocarbon solvent. The aromatic hydrocarbon solvent may contain one or more aromatic units. The slurries provide enhanced stability, safety, and do not degrade the solid-state electrolyte material.

### CROSS-REFERENCE TO RELATED APPLICATION

This application is related to and claims priority under 35 U.S.C. § 119 from U.S. Provisional Application No. 63/686,643 filed Aug. 23, 2024, titled “Slurry Containing an Aromatic Hydrocarbon, and Methods Thereof,” which is incorporated by reference herein for all purposes.

### FIELD OF THE DISCLOSURE

The present disclosure is directed toward methods of preparing slurries containing a solvent blend and binders. Therefore, the disclosure relates to the fields of batteries, including solid-state batteries, electronics, chemistry, and materials science.

### BACKGROUND AND INTRODUCTION

A solid-state electrochemical battery cell is constructed by assembling an interconnected system of positive electrodes or cathode layers, negative electrodes or anode layers, and separator layers, which may be solid electrolyte layers. Before these layers can be assembled into a cell, each layer must first be formed to a desired specification. A common method for forming these layers is first starting with a slurry containing a variety of materials including solvents, and then coating this slurry onto a substrate, and then drying it to remove the solvents. Before coating the slurries, they must first be mixed homogenously and then possess the proper rheological properties to coat the slurry on a substrate. The choice of solvent may be important in achieving the proper rheological properties. The choice of solvent is even more important in slurries that contain solid electrolytes, as the solvent may degrade the solid electrolyte material. In certain cases, the slurry should be used immediately after the slurry is formed to prevent substantial degradation of the electrolyte.

What is needed are methods for preparing slurries containing a solid electrolyte material, wherein the slurry achieves the desired rheological properties for coating the slurry on a substrate and subsequently drying the mixture while not degrading the solid electrolyte material, among other possible advantages and improvements.

It is with these observations in mind, among others, that aspects of the present disclosure were conceived.

### BRIEF DESCRIPTION OF THE FIGURES

The present disclosure may be understood by reference to the following detailed description taken in conjunction with the drawings briefly described below. It is noted that, for purposes of illustrative clarity, certain elements in the drawings may not be drawn to scale.

FIG. 1A shows an example process of the present disclosure for making solid-state electrochemical cell layers.

FIG. 1B shows an example process of the present disclosure for making solid-state electrochemical cell layers.

FIG. 1C shows an example process of the present disclosure for making solid-state electrochemical cell layers.

FIG. 2 shows an apparatus for making solid-state electrochemical cell layers using the processes described herein.

### SUMMARY

Provided herein are slurry compositions for use in preparing solid-state electrochemical cell layers. The slurry compositions include a solid electrolyte material and an aromatic hydrocarbon solvent. The aromatic hydrocarbon solvent may include an aromatic hydrocarbon solvent having one aromatic unit, an aromatic hydrocarbon solvent having two aromatic units, an aromatic hydrocarbon solvent having three aromatic units, or an aromatic hydrocarbon solvent having four aromatic units. In some embodiments, the aromatic hydrocarbon solvent has two or more aromatic units. In some embodiments, the aromatic hydrocarbon solvent is selected from the group consisting of a trimethylbenzene, a tetramethylbenzene, or a combination thereof. In some embodiments, the aromatic hydrocarbon solvent comprises an aromatic hydrocarbon solvent having two aromatic units and an aromatic hydrocarbon solvent having three or more aromatic units. In some embodiments, the aromatic hydrocarbon solvent comprises an aromatic hydrocarbon solvent having two aromatic units and an aromatic hydrocarbon solvent having one aromatic unit. The slurry may have a solids content from about 10% to less than 100%. The slurry may further comprise an alkane hydrocarbon solvent. The slurry may further comprise a binder, a conductive additive, an anode active material, a cathode active material, or any combination thereof.

Further provided herein is a method of making a solid-state electrochemical cell layer. The method includes dry mixing a solid electrolyte material, a binder, optionally a conductive additive, optionally an anode active material, and optionally a cathode active material to form a dry mixture; combining the dry mixture with an aromatic hydrocarbon solvent to form a slurry; coating the slurry onto a surface; drying the slurry to form a dry composition; and densifying the dry composition to form the solid-state electrochemical cell layer. In some embodiments, combining the dry mixture with the aromatic hydrocarbon solvent comprises milling the dry mixture and the aromatic hydrocarbon solvent in a high energy mill. In some embodiments, the method further includes laminating the solid-state electrochemical cell layer with a second dry composition. In some embodiments, combining the dry mixture with an aromatic hydrocarbon solvent comprises adding a first volume of the aromatic hydrocarbon solvent to the dry composition in an amount such that the amount of the aromatic hydrocarbon solvent present in mixture as compared to the total amount of binder in the mixture is greater than 0 wt % to 1 wt % and mixing; adding a second volume of the aromatic hydrocarbon solvent to the mixture such that the amount of solvent present in mixture as compared to the total amount of binder in the mixture is from about 1 wt % to about 10 wt % and mixing; and adding a third volume of the aromatic hydrocarbon solvent to the mixture such that the amount of solvent present in mixture as compared to the total amount of binder in the mixture is greater than 10 wt % and mixing.

### DETAILED DESCRIPTION

Before various aspects of the present invention are disclosed and described, it is to be understood that this invention is not limited to the particular methods, compositions, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity and in another example, a numerical range of “about 50 mg/mL to about 80 mg/mL” should also be understood to provide support for the range of “50 mg/mL to 80 mg/mL.”

In this disclosure, the terms “including,” “containing,” and/or “having” are understood to mean comprising, and are open ended terms.

Described herein are slurries and methods for making slurries containing a solid electrolyte material for use in making an electrochemical cell, which in some cases may be considered a solid-state battery cell. The inventors surprisingly discovered that the use of particular solvents in conjunction with one or more binders is especially effective at rapidly forming a slurry having desired rheological properties while preventing solid electrolyte degradation.

The slurry may comprise only aromatic hydrocarbon solvents. In other words, the solvent of the slurry may consist of one or more aromatic hydrocarbon solvents. The slurry may comprise an aromatic hydrocarbon solvent comprising one aromatic unit, two aromatic units, three aromatic units, four aromatic units, or any combination thereof. It should be understood that the meaning of “aromatic unit” as used herein encompasses an aromatic cyclical chain of carbons containing 3 or more carbons, 4 or more carbons, 5 or more carbons, and so on.

The aromatic hydrocarbon solvent may comprise a single aromatic unit such as xylenes, toluene, benzene, a trimethylbenzene, a tetramethylbenzene, or any combination thereof. In some examples, the trimethylbenzene may include 1,2,3-trimethylbenzene (hemimellitene), 1,2,4-trimethylbenzene (speudocumene), 1,3,5-trimethylbenzene (mesitylene), or any combination thereof. In some additional examples, the tetramethylbenzene may include 1,2,3,4-tetramethylbenzene (prehnitene), 1,2,3,5-tetramethylbenzene (isodurene), 1,2,4,5-tetramethylbenzene (durene), or any combination thereof.

The slurry may comprise an aromatic hydrocarbon solvent comprising two aromatic units such as biphenyl, naphthalene, or any combination thereof.

The slurry may comprise an aromatic hydrocarbon solvent comprising three aromatic units such as anthracene, phenalene, phenanthrene, fluorene, or any combination thereof.

The slurry may comprise an aromatic hydrocarbon solvent comprising 4 aromatic units such as pyrene, chrysene, tetracene, triphenylene, or any combination thereof.

The aromatic hydrocarbon solvent may contain 6-30 carbons, 6-24 carbons, 6-18 carbons, 6-14 carbons, 10-30 carbons, 10-24 carbons, 10-18 carbons, 10-14 carbons, 14-30 carbons, 14-24 carbons, 14-18 carbons, or any combination thereof.

The slurry may include a combination of aromatic hydrocarbon solvents having different numbers of aromatic units. For example, the slurry may include an aromatic hydrocarbon solvent having two aromatic units and an aromatic hydrocarbon solvent having three or more aromatic units. For example, the weight ratio of the aromatic hydrocarbon solvent with two aromatic units to the aromatic hydrocarbon solvent with three or more aromatic units may be from about 1:999 to about 1:90, about 1:999 to about 1:80, about 1:999 to about 1:70, about 1:999 to about 1:60, about 1:999 to about 1:50, about 1:999 to about 1:40, about 1:999 to about 1:30, about 1:999 to about 1:20, about 1:999 to about 1:10, about 1:90 to about 1:10, about 1:80 to about 1:10, about 1:70 to about 1:10, about 1:60 to about 1:10, about 1:50 to about 1:10, about 1:40 to about 1:10, about 1:30 to about 1:10, or about 1:20 to about 1:10. In some particular embodiments, the weight ratio of the aromatic hydrocarbon solvent with two aromatic units to the aromatic hydrocarbon solvent with three or more aromatic units may be from 1:999 to about 10:90, about 5:999 to about 5:95, or about 1:99 to about 3:97.

In another example, the slurry may include an aromatic hydrocarbon solvent having two aromatic units and an aromatic hydrocarbon solvent having one aromatic unit. The weight ratio of the aromatic hydrocarbon solvent with two aromatic units to the aromatic hydrocarbon solvent with one aromatic unit may be from about 1:999 to about 1:10. For example, the weight ratio of the aromatic hydrocarbon solvent with two aromatic units to the aromatic hydrocarbon solvent with one aromatic unit may be from about 1:999 to about 1:90, about 1:999 to about 1:80, about 1:999 to about 1:70, about 1:999 to about 1:60, about 1:999 to about 1:50, about 1:999 to about 1:40, about 1:999 to about 1:30, about 1:999 to about 1:20, about 1:999 to about 1:10, about 1:90 to about 1:10, about 1:80 to about 1:10, about 1:70 to about 1:10, about 1:60 to about 1:10, about 1:50 to about 1:10, about 1:40 to about 1:10, about 1:30 to about 1:10, or about 1:20 to about 1:10. In some particular embodiments, the weight ratio of the aromatic hydrocarbon solvent with two aromatic units to the aromatic hydrocarbon solvent with one aromatic unit may be from 1:999 to about 10:90, about 5:999 to about 5:95, or about 1:99 to about 3:97.

The aromatic hydrocarbon solvents may be preferred in some cases, as they enhance the stability of the slurry (e.g., particles in the slurry remain in suspension for extended periods of time after mixing), the safety of the process (e.g., using solvents with high flashpoint helps to prevent fires) all while not degrading the solid-state electrolyte material even at high temperatures.

The slurry may further comprise one or more alkane hydrocarbon solvents. The alkane hydrocarbon solvent may include heptane, octane, or isoparaffins containing from 9 to 20 carbons. The alkane hydrocarbon solvent may include branched alkanes or linear alkanes.

The slurry may be an electrode slurry. The electrode slurry may comprise an electrode active material (such as an anode active material or a cathode active material), a conductive additive, a solid-state electrolyte material, a binder, and a solvent as described above. Alternatively, the slurry may be a separator slurry. The separator slurry may comprise a solid-state electrolyte material, a binder, and a solvent as described above.

The slurry may further comprise a binder. In some embodiments, the binder may include fluororesin containing vinylidene fluoride (VdF), hexafluoropropylene (HFP), tetrafluoroethylene (TFE), and derivatives thereof as structural units. Specific examples thereof include homopolymers such as polyvinylidene fluoride (PVDF), polyhexafluoropropylene (PHFP), and polytetrafluoroethylene (PTFE), and binary copolymers such as copolymers of VdF and HFP such as poly (vinylene difluoride-hexafluoropropylene) copolymer (PVDF-HFP), and the like. In another embodiment, the binder may be one or more of a thermoplastic elastomer such as but not limited to styrene-butadiene rubber (SBR), styrene-butadiene-styrene copolymer (SBS), styrene-isoprene block copolymer (SIS), styrene-ethylene-butylene-styrene (SEBS), polyacrylonitrile (PAN), nitrile-butylene rubber (NBR), polybutadiene, polyisoprene, Poly (methacrylate) nitrile-butadiene rubber (PMMA-NBR) and the like. In a further embodiment, the binder may be one or more of an acrylic resin such as but not limited to polymethyl (meth) acrylate, polyethyl (meth) acrylate, polyisopropyl (meth) acrylate polyisobutyl (meth) acrylate, polybutyl (meth) acrylate, and the like. In yet another embodiment, the binder may be one or more of a polycondensation polymer such as but not limited to polyurea, polyamide paper, polyimide, polyester, and the like. In yet a further embodiment, the binder may be one or more of a nitrile rubber such as but not limited to acrylonitrile-butadiene rubber (ABR), polystyrene nitrile-butadiene rubber (PS-NBR), and mixtures thereof.

Preferably, the binder comprises a thermoplastic elastomer such as those comprising styrene and butadiene. For example, the binder may comprise styrene-butadiene-styrene copolymer (SBS), styrene-isoprene block copolymer (SIS), styrene-ethylene-butylene-styrene (SEBS), or combinations thereof. In some embodiments, the styrene content of the binder may be about 5% by weight (wt %) to about 50% by weight (wt %), such as from about 7 wt % to about 45 wt %, about 10 wt % to about 40 wt %, or about 12 wt % to about 35 wt %.

In some embodiments, the binder may comprise a high molecular weight binder and a low molecular weight binder. The high molecular weight binder may be the same species of binder as the low molecular weight binder, or it may be different. The high molecular weight binder has a longer polymer chain as compared to the low molecular weight binder. High molecular weight binders, as described herein, have a molecular weight of about 300,000 g/mol or higher. Low molecular weight binders, as described herein, have a molecular weight of about 100,000 or lower.

In embodiments wherein the slurry comprises a high molecular weight binder and a low molecular weight binder, the high molecular weight binder and the low molecular weight binder may be present in a weight ratio from about 10:90 to about 90:10, such as from about 10:90 to about 20:80, about 10:90 to about 30:70, about 10:90 to about 40:60, about 10:90 to about 50:50, about 10:90 to about 60:40, about 10:90 to about 70:30, about 10:90 to about 80:20, about 10:90 to about 90:10, about 20:80 to about 90:10, about 30:70 to about 90:10, about 40:60 to about 90:10, about 50:50 to about 90:10, about 60:40 to about 90:10, about 70:30 to about 90:10, about 80:20 to about 90:10, about 20:80 to about 80:20, about 25:75 to about 75:25, or about 30:70 to about 70:30.

The binder may be present in the slurry in an amount from about 1% to about 30% by weight. For example, the binder may be present in the slurry in an amount from about 1% to about 5%, about 1% to about 10%, about 1% to about 15%, about 1% to about 20%, about 1% to about 25%, about 1% to about 30%, about 5% to about 30%, about 10% to about 30%, about 15% to about 30%, about 20% to about 30%, about 25% to about 30%, about 5% to about 15%, about 5% to about 20%, about 10% to about 15%, about 10% to about 20%, or about 15% to about 20% by weight.

The slurry may further comprise a conductive additive. The conductive additive helps to evenly distribute the charge density throughout the anode. The conductive additives may include metal powders, fibers, filaments, or any other material known to conduct electrons. The conductive additive may comprise a carbon-based conductive additive, such as carbon fiber, graphite, graphene, carbon black, conductive carbon, amorphous carbon, vapor grown carbon fiber (VGCF), carbon nanotubes, carbon nanowires, activated carbon, and combinations thereof.

In some embodiments, the conductive additive may be present in the slurry in an amount from about 0% to about 15% by weight. In some aspects, the conductive additive may be present in the slurry in an amount from about 0% to about 10%, or about 0% to about 5% by weight. In some additional aspects, the conductive additive may be present in the slurry in an amount of about 0%, 1%, 2%, 3%, 4%, 5%, 6%, 7%, 8%, 9%, 10%, 11%, 12%, 13%, 14%, or about 15% by weight. In an example embodiment, the conductive additive is present in the slurry in an amount from about 0% to about 5% by weight.

In some embodiments, the average particle size of the conductive additive may be from about 5 nm to about 1000 nm. In some aspects, the average particle size of the conductive additive may be about from 5 nm to about 100 nm, about 5 nm to about 200 nm, about 5 nm to about 300 nm, about 5 nm to about 400 nm, about 5 nm to about 500 nm, about 5 nm to about 600 nm, about 5 nm to about 700 nm, about 5 nm to about 800 nm, about 5 nm to about 900 nm, about 100 nm to about 1000 nm, about 200 nm to about 1000 nm, about 300 nm to about 1000 nm, about 400 nm to about 1000 nm, about 500 nm to about 1000 nm, about 600 nm to about 1000 nm, about 700 nm to about 1000 nm, about 800 nm to about 1000 nm, about 900 nm to about 1000 nm, about 100 nm to about 500 nm, or about 200 nm to about 400 nm. In some embodiments, the conductive additive may have a particle size of about 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, or about 1000 nm. In some examples, the conductive additive may have an average particle size of about 30 nm. The average particle size (e.g., D₅₀) may be determined through any method known to those having ordinary skill in the art.

The slurry further comprises a solid electrolyte material. The solid electrolyte material may comprise an oxide, oxysulfide, sulfide, halide, nitride, or any other solid electrolyte known in the art. In some preferred embodiments, the solid electrolyte materials may comprise a sulfide solid electrolyte material, i.e., a solid electrolyte having at least one sulfur component. In some embodiments, the one or more solid electrolytes may comprise one or more material combinations such as Li₂S—P₂S₅, Li₂S—P₂S₅-LiI, Li₂S—P₂S₅—GeS₂, Li₂S—P₂S₅—Li₂O, Li₂S—P₂S₅—Li₂O-LiI, Li₂S—P₂S₅—LiI—LiBr, Li₂S—SiS₂, Li₂S—SiS₂—LiI, Li₂S—SiS₂—LiBr, Li₂S—S—SiS₂—LiCl, Li₂S—S—SiS₂—B₂S₃—LiI, Li₂S—S—SiS₂—P₂S₅—LiI, Li₂S—B₂S₃, Li₂S—P₂S₅—ZₘSₙ (where m and n are positive numbers, and Z is Ge, Zn or Ga), Li₂S—GeS₂, Li₂S—S—SiS₂—Li₃PO₄, and Li₂S—S—SiS₂—LiₓMO_{y} (where x and y are positive numbers, and M is P, Si, Ge, B, Al, Ga or In). Halide solid electrolytes may have the structure Li-M-X, M is a metal element, and X is a halogen. These can be expressed by the generic formula Li_{α}M⁴⁺_{β}N³⁺_{(1-β)}X_{Ω}Y_{(6-Ω)}, where: 0≤β≤1; 0≤Ω≤6; a=6−[(β*4)+(1−β)*3]; X and Y are a halogen such as F, Cl, Br, I; M is an element with an oxidation state of 4+ such as Ti, Zr, Hf, and Rf; and N is an element an oxidation state of 3+ such as Ga, In, and Tl, Sc, Y, Fe, Ru, Os, Er. Examples of halide electrolytes include Li₂ZrCl₆, Li₃InCl₆, Li_{2.25}Hf_{0.75}Fe_{0.25}Cl₄Br₂.

In another embodiment, the solid electrolyte material may be one or more of a Li₃PS₄, Li₄P₂S₆, Li₇P₃S₁₁, Li₁₀GeP₂S₁₂, Li₁₀SnP₂S₁₂. In a further embodiment, the solid electrolyte material may be one or more of a Li₆PS₅Cl, Li₆PS₅Br, Li₆PS₅I or expressed by the formula Li_{7-y}PS_{6-y}X_{y} where “X” represents at least one halogen and/or at least one pseudo-halogen, and where 0<y≤2.0 and where the halogen may be one or more of F, Cl, Br, I, and the pseudo-halogen may be one or more of N, NH, NH₂, NO, NO₂, BF₄, BH₄, AlH₄, CN, and SCN. In yet another embodiment, the solid electrolyte material be expressed by the formula Li_{8-y-z}P₂S_{9-y-z}X_{y}W_{z} (where “X” and “W” represents at least one halogen and/or at least one pseudo-halogen and where 0≤y≤1 and 0≤ z≤1) and where the halogen may be one or more of F, Cl, Br, I, and the pseudo-halogen may be one or more of N, NH, NH₂, NO, NO₂, BF₄, BH₄, AlH₄, CN, and SCN.

The solid-state electrolyte material may be present in the slurry in an amount from greater than 0% to about 60% by weight; for example, the solid-state electrolyte may be present in the slurry in an amount from greater than 0% to about 10% by weight, greater than 0% to about 20% by weight, greater than 0% to about 30% by weight, greater than 0% to about 40% by weight, greater than 0% to about 50% by weight, about 10% to about 60% by weight, about 20% to about 60% by weight, about 30% to about 60% by weight, about 40% to about 60% by weight, or about 50% to about 60% by weight. In some aspects, the solid-state electrolyte material may be present in the slurry in an amount of about 5%, 10%, 15%, 20%, 25%, 30%, 35%, 40%, 45%, 50%, 55%, or 60% by weight of the anode layer. In an example embodiment, the solid-state electrolyte material is present in the slurry in an amount from about 35% to about 45% by weight.

The solid-state electrolyte material may have an average particle size from about 0.5 microns to about 50 microns, such as from about 0.5 microns to about 1 micron, about 0.5 microns to about 10 microns, about 0.5 microns to about 20 microns, about 0.5 microns to about 30 microns, about 0.5 microns to about 40 microns, about 0.5 microns to about 0.5 microns, about 1 micron to about 50 microns, about 10 microns to about 50 microns, about 20 microns to about 50 microns, about 30 microns to about 50 microns, or about 40 microns to about 50 microns.

The slurry may further comprise an electrode active material, such as an anode active material or a cathode active material, either alone or in combination with the solid electrolyte material.

The electrode active material may be present in the slurry in an amount from about 30% to about 98% by weight of the slurry. In some aspects, the electrode active material may be present in the slurry in an amount of about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 30% to about 85%, about 30% to about 90%, about 30% to about 95%, about 35% to about 98%, about 40% to about 98%, about 45% to about 98%, about 50% to about 98%, about 55% to about 98%, about 60% to about 98%, about 65% to about 98%, about 70% to about 98%, about 75% to about 98%, about 80% to about 98%, about 85% to about 98%, about 90% to about 98%, about 40% to about 90%, about 40% to about 80%, about 40% to about 70%, about 40% to about 60%, about 40% to about 55%, about 40% to about 50%, or about 40% to about 45% by weight of the slurry.

In some embodiments, the electrode active material may comprise an anode active material. The anode active material preferably is an inorganic material. The anode active material may comprise one or more inorganic materials such as silicon (Si), silicon alloys, tin (Sn), tin alloys, germanium (Ge), germanium alloys, graphite, Li₄Ti₅O₁₂ (LTO) or other known anode active materials and combinations thereof.

In some embodiments, the electrode active material may comprise a cathode active material. The cathode active material may include nickel-manganese-cobalt (“NMC”) which can be expressed as Li(NiₐCo_{b}Mn_{c})O₂ (0<a<1, 0<b<1, 0<c<1, a+b+c=1) or, for example, NMC 111 (LiNi_{0.33}Mn_{0.33}Co_{0.33}O₂), NMC 433 (LiNi_{0.4}Mn_{0.3}Co_{0.3}O₂), NMC 532 (LiNi_{0.5}Mn_{0.3}Co_{0.2}O₂), NMC 622 (LiNi_{0.6}Mn_{0.2}Co_{0.2}O₂), NMC 811 (LiNi_{0.8}Mn_{0.1}Co_{0.1}O₂) or a combination thereof. In another embodiment, the cathode active material may comprise one or more of a coated or uncoated metal oxide, such as but not limited to V₂O₅, V₆O₁₃, MoO₃, LiCoO₂, LiNiO₂, LiMnO₂, LiMn₂O₄, LiNi_{1-Y}CO_{Y}O₂, LiCo_{1-Y}Mn_{Y}O₂, LiNi_{1-Y}Mn_{Y}O₂ (0≤Y<1), Li(NiₐCO_{b}Mn_{c}) O₄ (0<a<2, 0<b<2, 0<c<2, a+b+c=2), LiMn_{2-Z}Ni_{Z}O₄, LiMn_{2-Z}C_{Z}O₄ (0<Z<2), LiCoPO₄, LiFePO₄, CuO, Li(NiₐCo_{b}Al_{c}) O₂ (0<a<1, 0<b<1, 0<c<1, a+b+c=1) or a combination thereof. In yet another embodiment, the cathode active material may comprise one or more of a coated or uncoated metal sulfide such as but not limited to titanium sulfide (TiS₂), molybdenum sulfide (MoS₂), iron sulfide (FeS, FeS₂), copper sulfide (CuS), and nickel sulfide (Ni₃S₂) or combinations thereof. In still further embodiments, the cathode active material may comprise elemental sulfur(S). In additional embodiments, the cathode active material may comprise one or more of a fluoride cathode active material such as but not limited to lithium fluoride (LiF), sodium fluoride (NaF), calcium fluoride (CaF₂), magnesium fluoride (MgF₂), nickel (II) fluoride (NiF₂), iron (III) fluoride (FeF₃), vanadium (III) fluoride (VF₃), cobalt (III) fluoride (CoF₃), chromium (III) fluoride (CrF₃), manganese (III) fluoride (MnF₃), aluminum fluoride (AlF₃), and zirconium (IV) fluoride (ZrF₄), or combinations thereof.

The slurry of the present disclosure may have a solids content from about 10% to less than 100%. For example, the slurry may have a solids content from about 10% to about 20%, about 10% to about 30%, about 10% to about 40%, about 10% to about 50%, about 10% to about 60%, about 10% to about 70%, about 10% to about 80%, about 10% to about 90%, about 10% to less than 100%, about 20% to less than 100%, about 30% to less than 100%, about 40% to less than 100%, about 50% to less than 100%, about 60% to less than 100%, about 70% to less than 100%, about 80% to less than 100%, about 90% to less than 100%, about 50% to about 90%, about 60% to about 90%, or about 70% to about 90%.

The electrode slurry may have a viscosity from about 20 cP to about 3000 cP measured at a shear rate of about 100 s⁻¹. For example, the electrode slurry may have a viscosity from about 20 cP to about 100 cP, about 20 cP to about 500 cP, about 20 cP to about 1000 cP, about 20 cP to about 1500 cP, about 20 cP to about 2000 cP, about 20 cP to about 2500 cP, about 20 cP to bout 3000 cP, about 100 cP to about 3000 cP, about 500 cP to about 3000 cP, about 1000 cP to about 3000 cP, about 1500 cP to about 3000 cP, about 2000 cP to about 3000 cP, or about 2500 cP to about 3000 cP. In some embodiments, the electrode slurry may have a viscosity of about 20 cP, 50 cP, 100 cP, 150 cP, 200 cP, 250 cP, 300 cP, 350 cP, 400 cP, 450 cP, 500 cP, 550 cP, 600 cP, 650 cP, 700 cP, 750 cP, 800 cP, 850 cP, 900 cP, 950 cP, 1000 cP, 1100 cP, 1200 cP, 1300 cP, 1400 cP, 1500 cP, 1600 cP, 1700 cP, 1800 cP, 1900 cP, 2000 cP, 2100 cP, 2200 cP, 2300 cP, 2400 cP, 2500 cP, 2600 cP, 2700 cP, 2800 cP, 2900 cP, or about 3000 cP measured at a shear rate of about 100 s⁻¹.

The slurry of the present disclosure may be formed by combining an aromatic hydrocarbon solvent with one or more of a solid electrolyte material, a conductive additive, a binder, an anode active material, and a cathode active material.

In some embodiments, the slurry may be processed in stages where one or more of the aromatic hydrocarbon solvents is gradually added to the mixture of one or more of a solid electrolyte material, a conductive additive, or a binder.

At the first step, mixing one or more of the solid electrolyte material, the conductive additive, or the binder may be mixed without the addition of any solvents. This may be referred to as dry mixing. The dry mixing step may be performed for less than 1 minute up to about 1 hour.

After the dry mixing stage, the aromatic hydrocarbon solvent may be added along with any remaining ingredients to form the electrochemical cell. In one embodiment, the aromatic hydrocarbon solvent may be added such that the amount of solvent present in mixture is greater than 0 wt % to 1 wt %. Then, mixing may be performed for less than 1 minute up to less than 1 hour. Following this, more solvent may be added such that the amount of solvent present in mixture is from about 1 wt % to about 10 wt %. This mixing step may be performed for than 1 minute up to less than 1 hour. Following this, additional solvent may be added such that the amount of solvent present in mixture is greater than 10 wt %. This mixing step may be performed for more than 1 minute up to less than 48 hours.

The mixing of these materials may be performed by high energy milling techniques where the particle size of various material in the mixture are reduced. In some embodiments, the high energy milling may be accomplished using a vertical or horizontal ball mill (e.g., a planetary ball mill), tumble milling, an attritor mill, or other high energy mills known in the art.

The temperature of the materials during one or more of the mixing steps may be between 0° C. and 200° C. In some embodiments, the temperature of the materials during one or more of the mixing steps may be between 25° C. and 150° C., between 35° C. and 100° C., and between 45° C. and 70° C. In some embodiments, the temperature of the materials during one or more of the mixing steps may be greater than the boiling point of at least one of the aromatic solvents used in the slurry.

The combining and mixing steps may also be accomplished using stand mixing that utilize standard blending or mixing devices (e.g., a high shear mixer). Other methods of combining and mixing are generally known to those having ordinary skilled in the art and may be employed to produce a homogeneous slurry.

Further provided herein is an electrochemical cell layer comprising a solid-state electrolyte material, a binder, a conductive additive, a cathode active material, an anode active material, or any combination thereof. The electrochemical cell layers are made by casting any one of the slurries described above. Without wishing to be bound by theory, the solvents used herein do not react with solid electrolyte materials and their use leads to solid electrolyte materials and electrochemical cell layers containing fewer contaminants, such as materials containing Li—O or P—O.

The electrochemical cell layer may include one or more binders having a styrene content from about 5% by weight (wt %) to about 50% by weight (wt %) of the binder molecule. The styrene content of the binder may be about 7 wt % to about 45 wt %, about 10 wt % to about 40 wt %, or about 12 wt % to about 35 wt %. Without wishing to be bound by theory, greater dispersion of the solvent and binder in the mixture may be obtained by using a binder with the styrene content described due to interactions between the aromatic hydrocarbon solvent and the aromatic benzene ring in the styrene monomer.

The electrochemical cell layer may include an electrode layer or a separator layer. The electrode layer may include an electrode active material (i.e., an anode active material or a cathode active material), a solid-state electrolyte material, a high molecular weight binder, a low molecular weight binder, and optionally a conductive additive. The separator layer may include a solid-state electrolyte material, a high molecular weight binder, a low molecular weight binder, and optionally a conductive additive.

A separator layer made using the methods of the present disclosure may have an ionic conductivity of greater than about 0.15 mS. For example, the separator layer may have an ionic conductivity of greater than about 0.15 mS, greater than about 0.30 mS, greater than about 0.60 mS, or greater than about 1 mS.

The electrochemical cell layer may include any solid electrolyte material previously described herein. The electrochemical cell layer may include a solid electrolyte material in an amount from about 30% to about 99% by weight. In some aspects, the solid electrolyte material may be present in the electrochemical cell layer in an amount of about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 30% to about 85%, about 30% to about 90%, about 30% to about 95%, about 35% to about 99%, about 40% to about 99%, about 45% to about 99%, about 50% to about 99%, about 55% to about 99%, about 60% to about 99%, about 65% to about 99%, about 70% to about 99%, about 75% to about 99%, about 80% to about 99%, about 85% to about 99%, about 90% to about 99%, about 40% to about 90%, about 40% to about 80%, about 40% to about 70%, about 40% to about 60%, about 40% to about 55%, about 40% to about 50%, or about 40% to about 45% by weight.

When the electrochemical cell layer is a separator layer, the electrochemical cell layer may include a solid electrolyte material in an amount from about 60% to about 99% by weight. In some aspects, the solid electrolyte material may be present in the electrochemical cell layer in an amount of about 60% to about 65%, about 60% to about 70%, about 60% to about 75%, about 60% to about 80%, about 60% to about 85%, about 60% to about 90%, about 60% to about 95%, about 60% to about 99%, about 65% to about 99%, about 70% to about 99%, about 75% to about 99%, about 80% to about 99%, about 85% to about 99%, about 90% to about 99%, or about 70% to about 90% by weight.

The binder may be present in the electrochemical cell layer in an amount from about 1% to about 30% by weight. For example, the binder may be present in the electrochemical cell layer in an amount from about 1% to about 5%, about 1% to about 10%, about 1% to about 15%, about 1% to about 20%, about 1% to about 25%, about 1% to about 30%, about 5% to about 30%, about 10% to about 30%, about 15% to about 30%, about 20% to about 30%, about 25% to about 30%, about 5% to about 15%, about 5% to about 20%, about 10% to about 15%, about 10% to about 20%, or about 15% to about 20% by weight.

The conductive additive may be present in the electrochemical cell layer in an amount from about 0% to about 15% by weight. In some aspects, the conductive additive may be present in the electrochemical cell layer in an amount from about 0% to about 10%, or about 0% to about 5% by weight. In some additional aspects, the conductive additive may be present in the electrochemical cell layer in an amount of about 0%, 1%, 2%, 3%, 4%, 5%, 6%, 7%, 8%, 9%, 10%, 11%, 12%, 13%, 14%, or about 15% by weight. In an example embodiment, the conductive additive is present in the electrochemical cell layer in an amount from about 0% to about 5% by weight.

The electrode active material may be present in the electrochemical cell layer in an amount from about 30% to about 98% by weight. In some aspects, the electrode active material may be present in the electrochemical cell layer in an amount of about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 30% to about 85%, about 30% to about 90%, about 30% to about 95%, about 35% to about 98%, about 40% to about 98%, about 45% to about 98%, about 50% to about 98%, about 55% to about 98%, about 60% to about 98%, about 65% to about 98%, about 70% to about 98%, about 75% to about 98%, about 80% to about 98%, about 85% to about 98%, about 90% to about 98%, about 40% to about 90%, about 40% to about 80%, about 40% to about 70%, about 40% to about 60%, about 40% to about 55%, about 40% to about 50%, or about 40% to about 45% by weight.

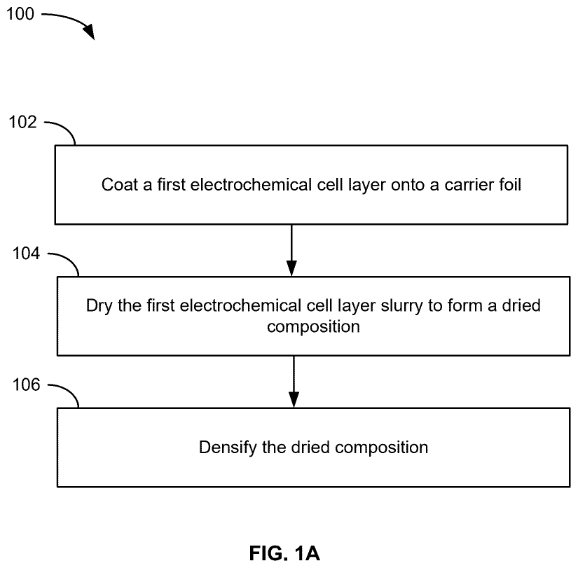

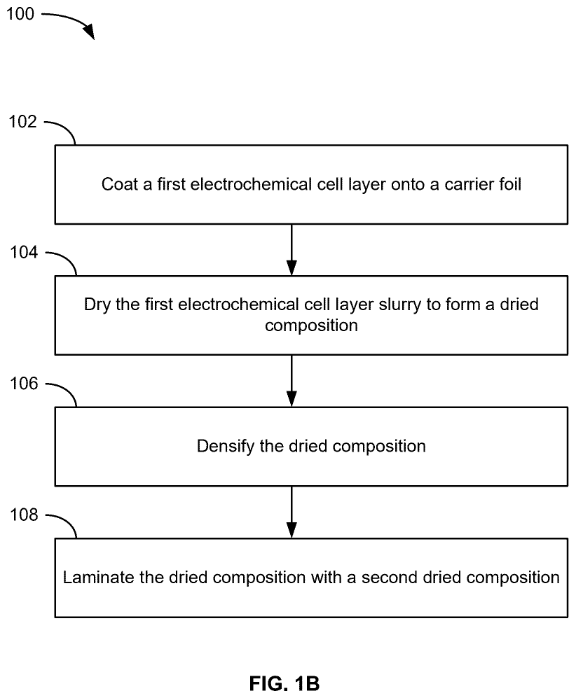

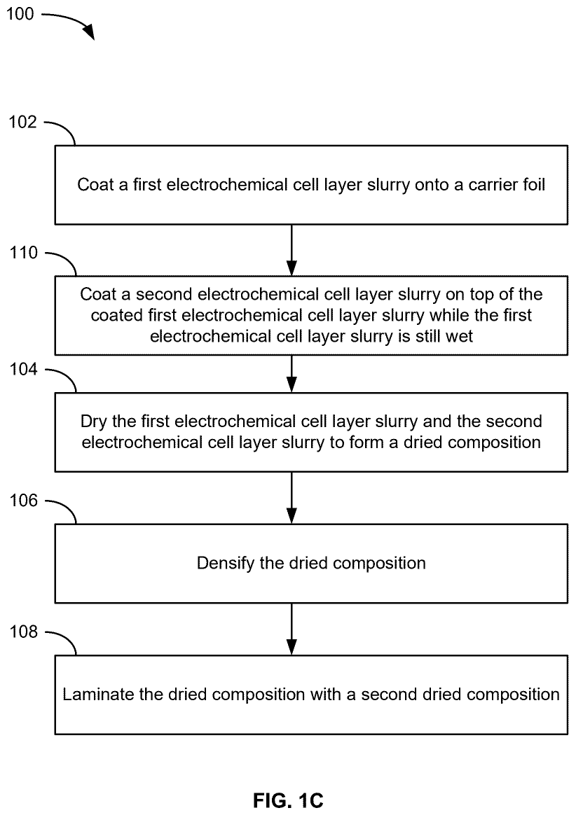

A process for preparing an electrochemical cell layer using a slurry of the present disclosure is shown in FIG. 1A. The process 100 begins at step 102 by coating the slurry onto a surface. The slurry may be any slurry described above, including an electrode slurry (e.g., an anode slurry or a cathode slurry), a separator slurry, or another slurry to make another electrochemical cell layer. The surface may comprise a carrier foil, a current collector, a dried electrochemical cell layer, or another surface. The coating may be accomplished by pouring the slurry onto a surface via gravity or by pumping the slurry onto the surface. The process may take place in ambient conditions, or may take place in an inert atmosphere such as nitrogen or argon. In some embodiments, the process may be conducted in an atmosphere comprising air and moisture. In other embodiments, the process may be conducted in an atmosphere comprising air and substantially no moisture (i.e., less than 1% humidity).

The slurry may be coated onto the surface at ambient temperature and pressure. In some aspects, the slurry may be coated onto the surface at a temperature up to the boiling point of the solvent system used in the slurry, or the slurry may be coated at cooler temperatures to limit vaporization of the solvent.

The process 100 continues at step 104 by drying the coated slurry to form a dried composition including at least one electrochemical cell layer. The drying at step 104 may occur at a temperature from about 15° C. to about 300° C. For example, the drying step 104 may occur at a temperature from about 15° C. to about 30° C., about 15° C. to about 50° C., about 15° C. to about 100° C., about 15° C. to about 150° C., about 15° C. to about 200° C., about 15° C. to about 250° C., about 15° C. to about 300° C., about 30° C. to about 300° C., about 50° C. to about 300° C., about 100° C. to about 300° C., about 150° C. to about 300° C., about 200° C. to about 300° C., or about 250° C. to about 300° C. In some embodiments, the drying step 106 may occur at a temperature of about 15° C., 20° C., 25° C., 30° C., 35° C., 40° C., 45° C., 50° C., 60° C., 70° C., 80° C., 90° C., 100° C., 125° C., 150° C., 175° C., 200° C., 225° C., 250° C., 275° C., or about 300° C.

After the drying step is completed, the amount of solvent left in the dried composition may range from 0.01% to 0% by weight of the composition. If there is solvent remaining in the dried composition, the electrochemical performance of an electrochemical cell containing this dried composite may be negatively affected.

The process 100 continues at step 106 by densifying the dried composition. The composition may be densified through densification processes known to those having ordinary skill in the art, such as calendaring, linear densification, compaction, or compression. In preferred embodiments, the densifying may be accomplished via calendering.

The dried composition may have a density after densification from about 50% to about 99% of the theoretical density of the composition. The theoretical density is defined as the maximum density of the composition that could be achieved assuming there are no voids or contaminants. The density may be from about 50% to about 60%, about 50% to about 70%, about 50% to about 80%, about 50% to about 90%, about 50% to about 95%, about 50% to about 99%, about 60% to about 99%, about 70% to about 99%, about 80% to about 99%, about 90% to about 99%, or about 95% to about 99% of the theoretical density of the dried composition.

The dried composition may have a porosity from about 1% to about 70%. For example, the dried composition may have a porosity from about 1% to about 10%, about 1% to about 20%, about 1% to about 30%, about 1% to about 40%, about 1% to about 50%, about 1% to about 60%, about 1% to about 70%, about 10% to about 70%, about 20% to about 70%, about 30% to about 70%, about 40% to about 70%, about 50% to about 70%, or about 60% to about 70%. The porosity of the dried composition may be measured through techniques known in the art, such as through SEM imaging, TEM imaging, FIB-SEM imaging, confocal microscopy, gas adsorption, mercury porosimetry, helium pycnometry, or other methods known in the art.

In another embodiment, as shown in FIG. 1B, the process 100 may further comprise step 108 of laminating the dried composition with a second dried composition. The lamination may be accomplished by lamination methods generally well known in the art, such as by calendar rolling. The lamination may further increase the density of the dried composition. The second dried composition may or may not contain a binder concentration gradient.

The second dried composition may comprise a second electrochemical cell layer. The second electrochemical cell layer may be a separator layer, an electrode layer, or another layer electrochemical cell layer. The second dried composition may be produced using the processes described herein, or it may be produced using a different process. Generally, the second dried composition may be individually calendered separately from the first dried composition.

FIG. 1C shows another embodiment of the process 100, which includes step 110 coating a second electrochemical cell layer slurry on top of the coated first electrochemical cell layer slurry while the first coated electrochemical cell layer slurry is still wet. The second electrochemical cell layer slurry may be a different type of electrochemical cell layer slurry as compared to the first electrochemical cell layer slurry. For example, if the first electrochemical cell layer slurry is a separator slurry, then the second electrochemical cell layer slurry may be an electrode slurry such as an anode slurry or a cathode slurry.

The process 100 proceeds as described above with respect to FIGS. 1A and 1B, wherein after the drying of step 104 and the densifying of step 106 are completed, the dried composition includes a first electrochemical cell layer and a second electrochemical cell layer. At step 108, the first dried composition (containing the first electrochemical cell layer and the second electrochemical cell layer) and the second dried composition (containing a third electrochemical cell layer) may be laminated together such that the second electrochemical cell layer is in contact with the third electrochemical cell layer; however, it will be appreciated by those having ordinary skill in the art that the first dried composition and the second dried composition may be laminated together such that the first electrochemical cell layer is in contact with the third electrochemical cell layer.

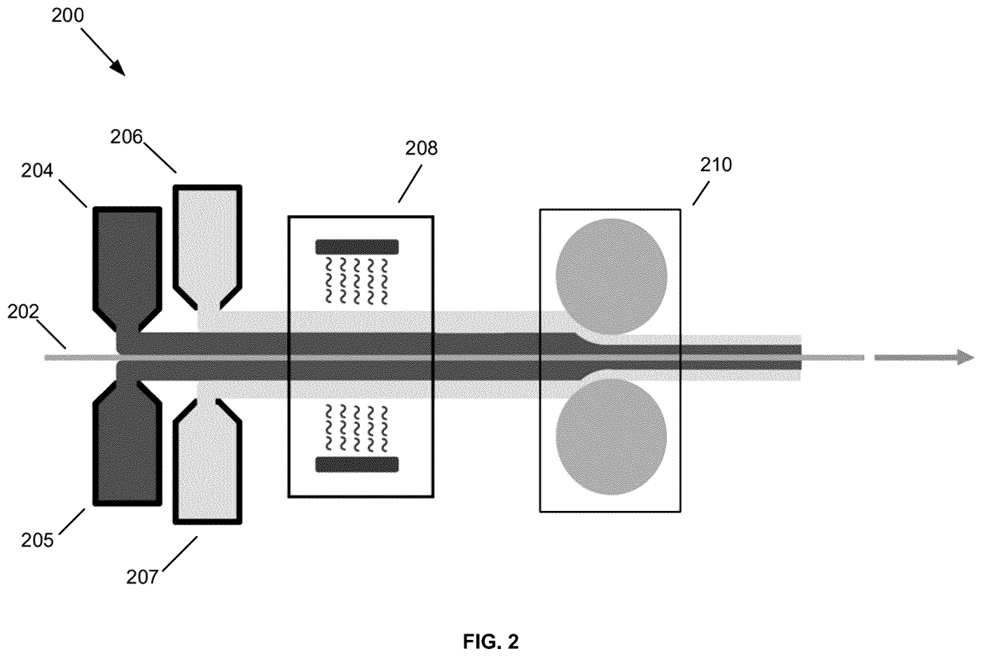

A depiction of an example process apparatus is shown in FIG. 2. Referring to FIG. 2, the apparatus 200 has two sides: Side A and Side B. The apparatus includes a surface 202, a first electrochemical cell layer slurry 204, a third electrochemical cell layer slurry 205, a second electrochemical cell layer slurry 206 a fourth electrochemical cell layer slurry 207, a drying module 208, and a densifying module 210. The two sides are oriented vertically, as shown in FIG. 2. Thus, the first electrochemical cell layer slurry 205 is deposited onto the surface 202 from below the surface 202. Likewise, the second electrochemical cell layer slurry 207 is deposited onto the surface 202 from below the surface 202. The inventors have surprisingly found that the slurries defined herein are capable of adhering to the surface 202 from below without falling or dripping from the surface 202. It will be appreciated by those having skill in the art that the process may be performed using only one of the sides; however, for efficiency and maximizing output, use of both sides is preferred.

The surface 202 may comprise a carrier foil or a current collector. The carrier foil or the current collector may comprise one or more of copper, aluminum, nickel, titanium, stainless steel, magnesium, iron, zinc, indium, germanium, silver, platinum, or gold. In some embodiments, the current collector may have a thickness from about 5 μm to about 10 μm. In some embodiments, the current collector includes a carbon coating. In preferred embodiments, the current collector comprises copper, nickel, and/or steel. Alternatively, the surface 402 may comprise a dried electrochemical cell layer.

The apparatus 200 shown in FIG. 2 may be used in a process for producing electrochemical cells as described herein. The process generally includes simultaneously coating a top side and a bottom side of a two-sided surface with a first wet electrode slurry and a second wet electrode slurry, respectively; and, simultaneously coating a first separator slurry and a second separator slurry on each of the respective top and bottom coated wet electrode slurry resulting in two parallel regions of wet-on-wet areas of contact between electrode and separator to physically form two neighboring electrochemical cell portions of a solid state battery. The first and second wet electrode slurries may each be any of the electrode slurries described herein. The first and second separator slurries may each be any of the separator slurries described herein. The two-sided surface may be any of the surfaces provided herein.

What is claimed is:

1. A slurry comprising a solid electrolyte material and an aromatic hydrocarbon solvent having two or more aromatic units.

2. The slurry of claim 1, wherein the aromatic hydrocarbon solvent comprises an aromatic solvent having two aromatic units.

3. The slurry of claim 2, wherein the aromatic hydrocarbon solvent comprises biphenyl, naphthalene, or any combination thereof.

4. The slurry of claim 1, wherein the aromatic hydrocarbon solvent comprises an aromatic solvent having three aromatic units.

5. The slurry of claim 4, wherein the aromatic hydrocarbon solvent comprises anthracene, phenalene, phenanthrene, fluorene, or any combination thereof.

6. The slurry of claim 1, wherein the aromatic hydrocarbon solvent comprises an aromatic solvent having 4 aromatic units.

7. The slurry of claim 6, wherein the aromatic hydrocarbon solvent comprises pyrene, chrysene, tetracene, triphenylene, or any combination thereof.

8. The slurry of claim 1, wherein the aromatic hydrocarbon solvent comprises an aromatic hydrocarbon solvent having two aromatic units and an aromatic hydrocarbon solvent having three or more aromatic units.

9. The slurry of claim 8, wherein the weight ratio of the aromatic hydrocarbon solvent having two aromatic units and the aromatic hydrocarbon solvent having three or more aromatic units is from about 1:999 to about 1:10.

10. The slurry of claim 1, wherein the aromatic hydrocarbon solvent comprises an aromatic hydrocarbon solvent having two aromatic units and an aromatic hydrocarbon solvent having one aromatic unit.

11. The slurry of claim 1, wherein the slurry has a solids content from about 10% to less than 100%.

12. The slurry of claim 1, wherein the aromatic hydrocarbon solvent comprises 6-30 carbons.

13. The slurry of claim 1, further comprising a binder.

14. The slurry of claim 1, further comprising an alkane hydrocarbon solvent.

15. The slurry of claim 13, wherein the alkane hydrocarbon solvent comprises heptane, octane, isoparaffins containing from 9-20 carbons, or any combination thereof.

16. The slurry of claim 1, further comprising a conductive additive.

17. The slurry of claim 1, further comprising a cathode active material or an anode active material.

18. A slurry comprising a solid electrolyte material and an aromatic hydrocarbon solvent selected from the group consisting of a trimethylbenzene, a tetramethylbenzene, or a combination thereof.

19. A method of making a solid-state electrochemical cell layer comprising:
dry mixing a solid electrolyte material, a binder, optionally a conductive additive, optionally an anode active material, and optionally a cathode active material to form a dry mixture;
combining the dry mixture with an aromatic hydrocarbon solvent to form a slurry;
coating the slurry onto a surface;
drying the slurry to form a dry composition; and
densifying the dry composition to form the solid-state electrochemical cell layer.

20. The method of claim 19, wherein combining the dry mixture with the aromatic hydrocarbon solvent comprises milling the dry mixture and the aromatic hydrocarbon solvent in a high energy mill.

21. The method of claim 19, further comprising laminating the solid-state electrochemical cell layer with a second dry composition.

22. The method of claim 19, wherein combining the dry mixture with an aromatic hydrocarbon solvent comprises:
adding a first volume of the aromatic hydrocarbon solvent to the dry composition in an amount such that the amount of the aromatic hydrocarbon solvent present in mixture as compared to the total amount of binder in the mixture is greater than 0 wt % to 1 wt % and mixing;
adding a second volume of the aromatic hydrocarbon solvent to the mixture such that the amount of solvent present in mixture as compared to the total amount of binder in the mixture is from about 1 wt % to about 10 wt % and mixing; and
adding a third volume of the aromatic hydrocarbon solvent to the mixture such that the amount of solvent present in mixture as compared to the total amount of binder in the mixture is greater than 10 wt % and mixing.
